(19) United States
(12) Patent Application Publication
(10) Pub. No.: US 2010/0136705 A1
(43) Pub. Date: Jun. 3, 2010

(54) METHOD FOR MEASURING CONCENTRATION OF PEROXYCARBOXYLIC ACID AND APPARATUS THEREFOR

(76) Inventors: Sachiko Kojima, Osaka (JP); Taro Furuta, Osaka (JP); Toshio Kasai, Osaka (JP); Dock-Chil Che, Osaka (JP)

Correspondence Address: WENDEROTH, LIND & PONACK, L.L.P., 1030 15th Street, N.W.,, Suite 400 East, Washington, DC, 20005-1503, US

(21) Appl. No.: 12/451,046

(22) PCT Filed: Apr. 25, 2008
(86) PCT No.: PCT/JP2008/058024

§ 371 Date: Jan. 7, 2010

(30) Foreign Application Priority Data
Apr. 25, 2007 (JP) 2007-141053

(51) Int. Cl. G01N 33/00

(52) U.S. Cl. 436/129

(57) ABSTRACT

A method for measuring only a concentration of a peroxycarboxylic acid in an equilibrium mixture containing peroxycarboxylic acid and hydrogen peroxide, comprising the following steps a) and b); a) adding potassium iodide to the equilibrium mixture to cause the generation of iodine and providing the resulting mixture as a measurement sample; and b) measuring the amount of light that goes through the measurement sample to determine only the concentration of a peroxycarboxylic acid.

### TECHNICAL FIELD

The present invention relates to a method for measuring only the concentration of a peroxycarboxylic acid in an equilibrium mixture containing the peroxycarboxylic acid and hydrogen peroxide and an apparatus used for the measurement.

### BACKGROUND ART

Equilibrium mixtures containing peroxycarboxylic acid (specifically, peracetic acid) and hydrogen peroxide are utilized in broad areas such as various oxidation reactions, and sterilization in medical treatment, food, environment area and the like. Especially, when it is used for the purpose of sterilization, a lower limit of its concentration for use is defined in many cases, in order to keep the availability thereof. However, the concentration of peroxycarboxylic acid is reduced with time, since peroxycarboxylic acid, compound is unstable in general. Thus it is desirable that exact concentration of peroxycarboxylic is always recognized.

In many cases, peroxycarboxylic acid exists as an equilibrium mixture of peroxycarboxylic acid and carboxylic acid, hydrogen peroxide, water. Further, the two peroxide compounds, peroxycarboxylic acid and hydrogen peroxide have similar characteristics (i.e. oxidizability) to each other. Therefore, the fractionation and the quantitative determination between the two peroxide compounds are considerably difficult.

As currently-used methods for quantitative determination, there exist the following methods. As a titration method there is a method in which, by utilizing the difference in oxidizability between peroxycarboxylic acid and hydrogen peroxide, fractionating hydrogen peroxide and determining quantity thereof by using cerium sulfate or permanganic acid potassium, and fractionating peroxycarboxylic acid (such as peracetic acid) and determining quantity thereof by using sodium thiosulfate. However, in this method, it is impossible to determine quantities of both peroxide compounds at the same time.

Moreover, known is also a quantitative determination method using the fact that the rate of reaction between a percarboxylic acid and potassium iodide is different from that of a reaction between hydrogen peroxide and potassium iodide. This method is a method of reducing iodine generated by the addition of potassium iodide with sodium thiosulfate. It is reported that a percarboxylic acid and hydrogen peroxide can be fractionally and quantitatively determined at the same time from a relationship between any two times and the amount of sodium thiosulfate necessary for reducing iodine generated at each of the times. Hereinafter, this will be called iodometric titration (see, for example, Non-Patent Document 1). Suggested is also a titrating method wherein an attempt is made for improving this method (see Patent Document 1). However, in these methods, the manner for measuring operations is complicated, and an exclusive tool or space is also required. Moreover, it takes much time to obtain measurement results. Thus, it is difficult to say that these methods are simple methods. There also remains a problem that it is necessary to use a compound regulated about the discharge amount thereof into the environment (PRTR material), such as molybdenum or manganese.

Furthermore, suggested are also quantitative determination methods which use an electrochemical technique to determine a percarboxylic acid and hydrogen peroxide fractionally and quantitatively at the same time by potentiometric titration (see, for example,. Patent Documents 2 and 3). However, these methods have a difficulty that the apparatus therefor is large in scale and expensive in many cases.

The method that is most inexpensive and simplest and is used in many actual locations is a method using a test paper. This is a method of immersing a test paper to which a color-developing agent is fixed into a solution to be examined, and deciding the concentration in accordance with the color development degree thereof. However, the decision is based on color sense; accordingly, the concentration cannot be precisely obtained as a numerical value. Moreover, the criterion of the decision is vague; thus, there is often caused a problem that results of the decision are varied depending on testing persons.

Patent Document 1: Japanese Patent No. 3170526

Patent Document 2: Japanese Patent No. 3813606

Patent Document 3: Japanese Patent Laid-Open Publication No. 2006-242629

Non-Patent Document 1: The Analyst, published by Royal Society of Chemistry, August in 1962, vol. 87, p. 653

### DISCLOSURE OF THE INVENTION

### Problems to be Solved by the Invention

As described above, some methods for measuring the concentration of a percarboxylic acid are known; however, a simple method for measuring the concentration rapidly and precisely has not been found easily in the actual circumstances.

Incidentally, in the case of an equilibrium mixture of a percarboxylic acid and hydrogen peroxide, an effective component therein is the percarboxylic acid in many articles wherein the mixture is used. Thus, a main object of the present invention is to provide a method for determining only the concentration of a percarboxylic acid easily, rapidly and precisely in an equilibrium mixture of the percarboxylic acid and hydrogen peroxide, and a measuring apparatus used in this measuring method.

### Means for Solving the Problems

In order to solve the problems, the measuring method according to the present invention is characterized by adding potassium iodide to an equilibrium mixture containing a percarboxylic acid and hydrogen peroxide, thereby generating iodine, and then measuring the amount of light that goes through this mixture, thereby determining the concentration of the percarboxylic acid quantitatively.

In the invention, the concentration of the percarboxylic acid in the measurement sample is preferably from 0.01 to 50 ppm. However, even a solution having a percarboxylic acid concentration not less than this range may be used in the state that the solution is diluted to set the concentration of the percarboxylic acid into this concentration range. Outside this range, the error of the measured value is large so that a difference thereof from an actual concentration of the percarboxylic acid may be generated.

In the invention, the pH value of the measurement sample is preferably within a range of 1<pH<6. If the pH is 6 or more, the amount of generated iodine is gradually reduced. On the other hand, if the pH is 1 or less, hydrogen peroxide, which exists together, reacts with potassium iodide to generate iodine, so that the amount of iodine is gradually increased. It therefore becomes difficult to obtain a precise concentration of the percarboxylic acid.

In the invention, the amount of potassium iodide in the measurement sample is preferably from 2 to 60 times the number of moles of the percarboxylic acid, more preferably from 3 to 30 times the number, and most preferably from 3 to 15 times the number. If the amount of potassium iodide in the measurement sample is less than 2 times the number of moles of the percarboxylic acid, potassium iodide is insufficient for the amount required for the reaction of potassium iodide with the percarboxylic acid. On the other hand, if the amount is more than 60 times, hydrogen peroxide, which exists together, reacts with potassium iodide to generate iodine, so that the amount of iodine is gradually increased. It therefore becomes difficult to obtain a precise concentration of the percarboxylic acid.

In the invention, the wavelength of the light used in the measurement is preferably within a range of 440 to 600 nm. If the wavelength is shorter than 440 nm, the peak overlaps with a peak of a polyiodide ion having an absorption maximum near 350 nm. On the other hand, if the wavelength is longer than 600 nm, the light is weakly absorbed. As a result, it may become difficult to obtain a precise concentration of the percarboxylic acid.

The percarboxylic acid that can be quantitatively determined by the method of the invention may be any percarboxylic acid that is reactive with potassium iodide to generate iodine rapidly. Of such percarboxylic acids, peracetic acid is most widely used, and the concentration of peracetic acid can be quantitatively determined easily and rapidly by the method of the invention.

Additionally, a small-sized and inexpensive apparatus for measuring the concentration of a percarboxylic acid can be realized by using a light-emitting diode (LED) as a light source, detecting transmitted light that is emitted from this light source and goes through a measurement sample by means of a photodiode, and then obtaining the concentration of the percarboxylic acid therein on the basis of the detected result.

### Effects of the Invention

According to the invention, only the concentration of a percarboxylic acid can be precisely measured in an equilibrium mixture of the percarboxylic acid and hydrogen peroxide without receiving an effect of hydrogen peroxide. Moreover, the concentration can be quantitatively determined easily and rapidly by use of a small sample amount since the measurement is made with reference to the light amount. Furthermore, from a minute amount of potassium iodide, iodine is generated to color the sample; therefore, an especial color developing agent or reagent as in the prior art is not required. Thus, an analyzing method clean from an environmental viewpoint can be provided.

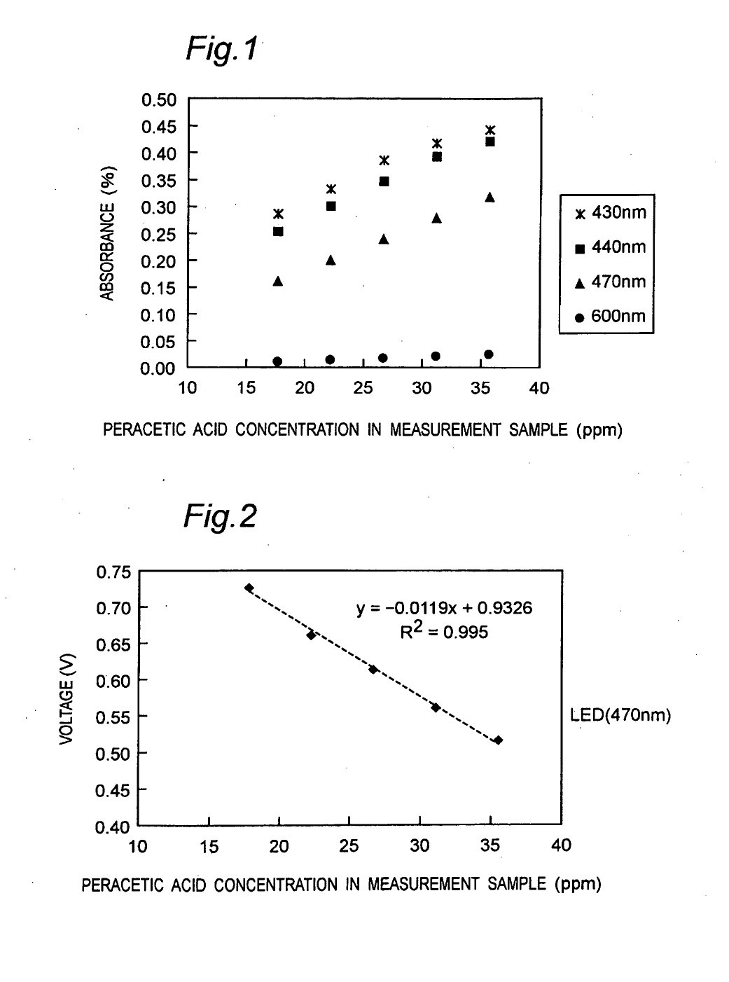

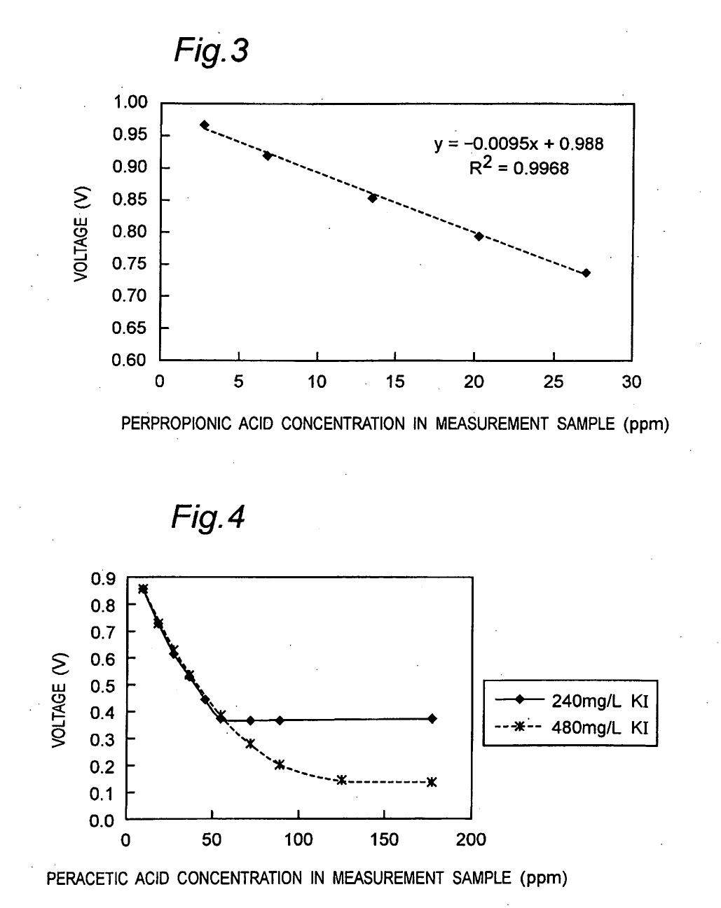

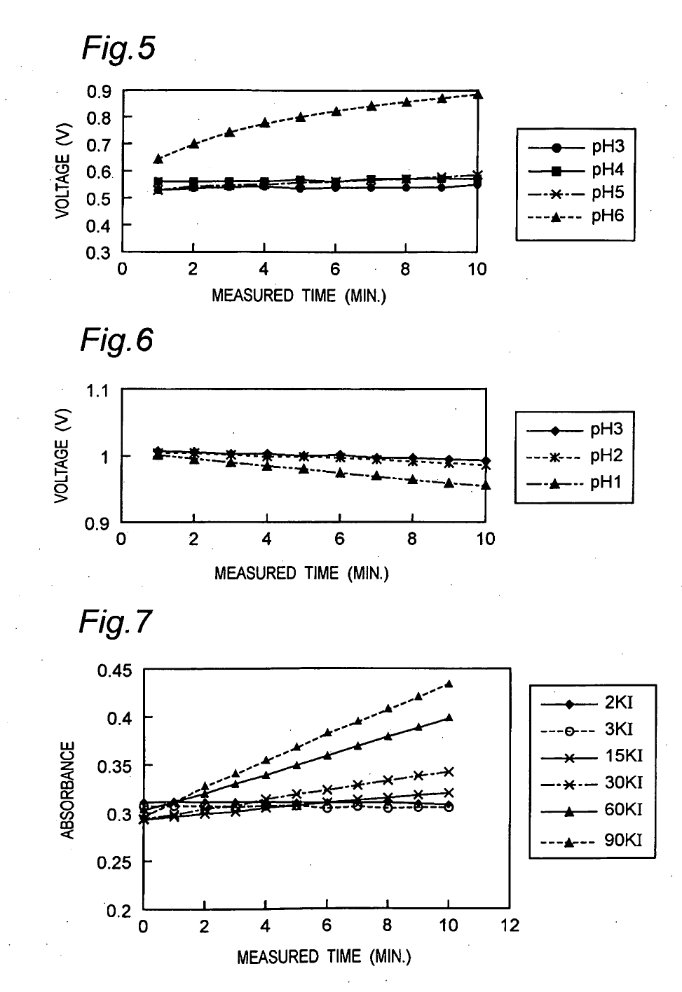

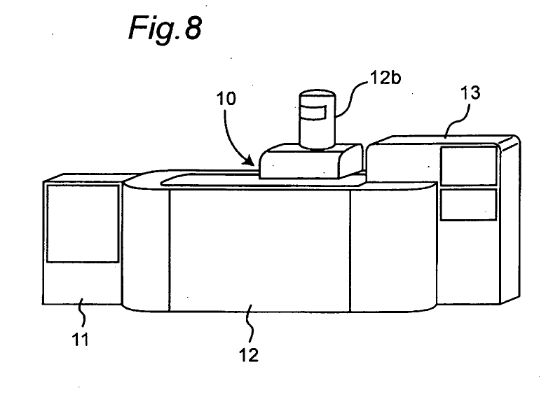

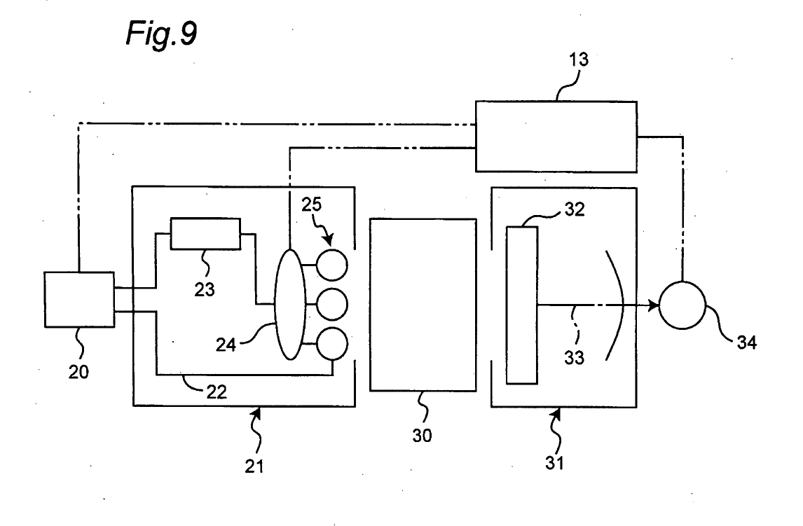

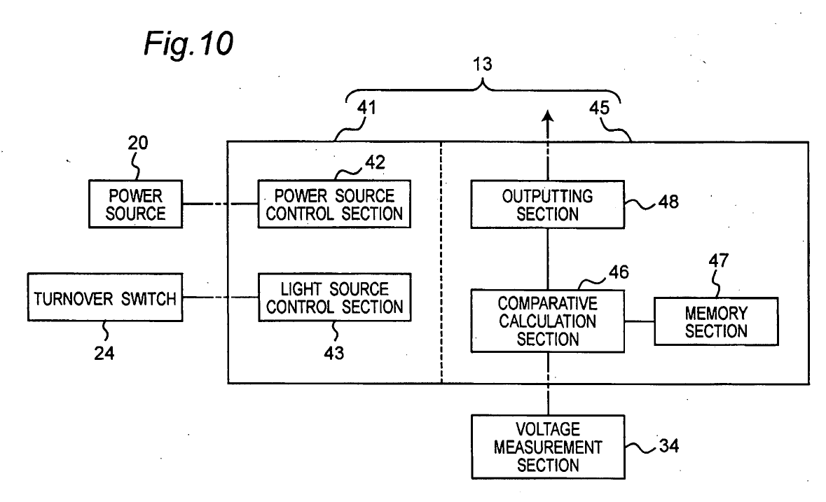

### BRIEF DESCRIPTION OF THE DRAWINGS

[FIG. 1] The drawing is a graph showing a correspondence between the concentration of peracetic acid in a measurement sample calculated by iodometric titration and the absorbance at each of 440, 470 and 600 nm according to an ultraviolet-visible spectrophotometer in Example 1.

[FIG. 2] The drawing is a graph showing a correspondence between the concentration of peracetic acid in a measurement sample calculated by iodometric titration and the voltage measured by a 470-nm wavelength LED/photodiode method in Example 1.

[FIG. 3] The drawing is a graph showing a correspondence between the concentration of perpropionic acid in a measurement sample calculated by iodometric titration and the voltage measured by a 470-nm wavelength LED/photodiode method in Example 4.

[FIG. 4] The drawing is a graph showing a correspondence between the concentration of peracetic acid in a measurement sample calculated by iodometric titration and the voltage measured by a 470-nm wavelength LED/photodiode method in Example 5.

[FIG. 5] The drawing is a graph showing a change with time in the measured voltage when each of potassium iodide solutions having pHs of 3, 6 and values therebetween, respectively, was used to measure peracetic acid in Example 6.

[FIG. 6] The drawing is a graph showing a change with time in the measured voltage when each of potassium iodide solutions having pHs of 1, 3 and a value therebetween, respectively, was used to measure hydrogen peroxide in Example 6.

[FIG. 7] The drawing is a graph showing a change with time in the absorbance when peracetic acid was measured while the concentration of a potassium iodide solution was varied in Example 7.

[FIG. 8] The drawing is a perspective view of the whole of a measuring apparatus used to measure the concentration of a percarboxylic acid according to an embodiment of the invention.

[FIG. 9] The drawing is an explanatory view which schematically illustrates a main portion of the measuring apparatus.

[FIG. 10] The drawing is a block diagram which schematically illustrates the structure of a control analysis section of the measuring apparatus.

### DESCRIPTION OF THE REFERENCE NUMERALS

10 apparatus for measuring the concentration of a percarboxylic acid

11 driving force section

12 measuring section

13 control analysis unit

20 power source

21 light-emitting section

24 turnover switch

25 light-emitting elements (LEDs: light-emitting diodes)

30 sample container

31 light-receiving section

32 light-receiving element (photodiode)

34 voltage measurement section

41 control section

45 analysis section

46 comparative calculation section

47 memory section

### BEST MODE FOR CARRYING OUT THE INVENTION

An embodiment of the method for measuring the concentration of a percarboxylic acid according to the present invention will be described hereinafter.

Previous to the description by way of working examples, a measuring apparatus for carrying out the measuring method of the invention is first described.

FIG. 8 is a perspective view of the whole of the measuring apparatus, which is used to measure the concentration of a percarboxylic acid according to the present embodiment, and FIG. 9 is an explanatory view which schematically illustrates a main portion of the measuring apparatus.

As illustrated in these drawings, an apparatus 10, which is the measuring apparatus according to the embodiment, has, as main constituting elements thereof, a driving force section 11 which is equipped with a power source 20 (or is to be connected to an external power source), a measuring section 12 equipped with an injection region 12b in which a measurement sample (equilibrium mixture containing a percarboxylic acid and hydrogen peroxide) is to be injected, and a control analysis unit 13 for controlling the driving force section 11 and the measuring section 12 and further determining the concentration of the measurement sample quantitatively.

The measuring section 12 is equipped with a sample container 30, the form of which is, for example, cylindrical, for containing the measurement sample injected from the injection region 12b, a light-emitting section 21 arranged on one side (the left side in FIG. 9) of the sample container 30, and a light-receiving section 31 arranged oppositely to the light-emitting section 21 so as to interpose the sample container 30 therebetween. The sample container 30 is preferably made of a material having chemical resistance and a high light transmissibility, and is made of, for example, glass in the embodiment. Instead of glass, any material may be used as far as the material has light transmissibility and chemical resistance to some degree or more, examples thereof being acrylic resin, vinyl chloride resin and PET resin.

The light-emitting section 21 is a section for emitting light of a predetermined wavelength toward the measurement sample contained in the sample container 30, and has plural light sources 25 (light-emitting elements) connected through a light-emitting circuit 22 to the power source 20. These light-emitting elements 25 are made of, for example, high-illuminance LEDs (light-emitting diodes) which emit light rays having different wavelengths, respectively, and are preferably arranged in parallel to the longitudinal axis of the sample container 30. The light-emitting circuit 22 has a predetermined resistor 23 and a changeover switch 24 (for example, a known rotary switch). The light-emitting elements 25 are each connected switchably to the changeover switch 24. In accordance with a control signal from the control analysis unit 13, one or more elements used as one or more light sources, out of the light-emitting elements 25, can be switched.

The light-receiving section 31 is a section for receiving light that is emitted from the light-emitting section 21 and goes through the sample container 30 and the measurement sample therein, and is equipped with a light-receiving element 32 made of, for example, a silicon (Si) photodiode. This light-receiving element 32 is preferably arranged in parallel to the longitudinal axis of the sample container 30 and over a scope in which transmitted light from all of the light-emitting elements 25, each of which is used as a light source, can be received. A voltage measurement section 34 is connected through a light-receiving circuit 33 to the light-receiving element 32, the section 34 being a section for measuring a voltage generated when light which goes through the sample container 30 and the measurement sample is received by the photodiode. Any measured result of the voltage measurement section 33 is inputted, as a signal, into the control analysis unit 13. This voltage measurement section 34 is a section using a structure known in the prior art for detecting a voltage change when a light-receiving sensor receives light.

FIG. 10 is a block diagram which schematically illustrates the structure of the control analysis unit 13. As illustrated in this diagram, the control analysis unit 13 has a power source control section 42 for controlling the turning-on and turning-off of the power source 20, the electric power amount to be supplied, and others, and a control section 41 having a light source control section 43 for controlling the changeover state of the light sources (light-emitting elements 25) through the changeover switch, and others. The control analysis unit 13 has an analysis section 45 having a comparative calculation section 46 into which any measured result from the voltage measurement section 34 is inputted as a signal. The control analysis unit 13 is preferably made mainly of a microcomputer, and is connected to the power source 20, the changeover switch 24, the voltage measurement section 34 and others in such a manner that the unit 13 can receive signals therefrom and can give signals thereto.

The analysis section 45 has a memory section 47 connected to the comparative calculation section 46 in such a manner that the section 46 can receive signals from the section 47 and can give signals to the section 47. The following correlation data about each of light rays having different wavelengths is memorized in the memory section 47 so as to be readable: a correlation data between the voltage value generated when the light which goes through each of various measurement samples is received by the photodiode and the concentration. This memory section 47 may be set, as an external memory, outside the control analysis unit 13.

When a measured data from the voltage measurement section 34 is inputted, as a signal, into the comparative calculation section 46, the correlation data on the measuring target sample are read out and then the correlation data and the measured data from the voltage measurement section 34 are compared with each other to make a required calculation. In this way, the concentration of the sample to be measured can be obtained. The thus-obtained concentration can be outputted through an outputting section 48 to a display section (not illustrated) of the measuring apparatus 10, such as a display thereof, or a printing device (not illustrated), such as a printer.

With reference to working examples, the following will describe the measurement of a percarboxylic acid concentration which was made by use of the measuring apparatus 10.

### Example 1

In Example 1, a 6%-peracetic acid antiseptic solution (trade name: ACECIDE manufactured by Saraya Co., Ltd. was diluted 20 times with distilled water, and the resultant was used as a test solution. The concentration of peracetic acid in the test solution was obtained by iodometric titration. As a result, the concentration of peracetic acid was 0.356%. To each of 0.1 mL, 0.2 mL and volumes therebetween of this test solution was added 240 mg/L of a potassium iodide solution to set the total volume to 20 mL (measurement samples). This was blended, and then light rays having wavelengths of 430 nm, 440 nm, 470 nm and 600 nm, respectively, were each used to measure the absorbance (of each of the samples). The measured results are shown in FIG. 1. The absorbances were measured with a known ultraviolet-visible spectrophotometer.

In FIG. 2 are shown results obtained by measuring the voltage generated when one of the light-emitting elements 25 (LEDs) having a wavelength of 470 nm was used as a light source in the measuring apparatus 10 and light going through each of the measurement samples was received by the light-receiving element 32 (photodiode).

Regression formulae corresponding to the measured results shown in FIG. 1 and the correlation coefficient R² of each of the formulae are as follows:

Wavelength of 430 nm: y=1.5636x+0.1389 (R²=0.9758)

Wavelength of 440 nm: y=1.6740x+0.0909 (R²=0.9927)

Wavelength of 470 nm: y=1.5436x+0.0079 (R²=0.9978)

Wavelength of 600 nm: y=0.0928x−0.0009 (R²=0.9980)

As described above, the correlation coefficient R² of the regression formulae corresponding to the measured results in FIG. 1 is 0.9927 when the wavelength is 440 nm, 0.9978 when it is 470 nm, and 0.9980 when it is 600 nm. It is understood that in these cases, an excellent linearity can be obtained; however, when the wavelength is 430 nm, R² is 0.9758 so that the linearity is declined.

In the meantime, as shown in FIG. 2, in the measuring apparatus 10 of the present embodiment also, wherein the light-emitting elements 25 (LEDs) and the light-receiving element 32 (photodiode) were used, the correlation coefficient R² of the regression formula corresponding to the measured results is 0.9950. Thus, it was verified that, between the peracetic acid concentration in the measurement samples and the measured results, an excellent linear relationship is obtained in the same manner as in the case of the absorbances (see FIG. 1).

Accordingly, it is advisable to use the measuring apparatus 10 of the embodiment; collect a correlation data showing a relationship between the voltage value generated when light going through each of various measurement samples (equilibrium mixture of hydrogen peroxide and each of percarboxylic acids) is received by the photodiode 32 about each of the light sources 25 (light-emitting diodes: LEDs) having the difference wavelengths, respectively, and the concentration; store the data, the number of which is large, into the memory section 47; read out the corresponding data in accordance with the species of a measurement sample and the used light source; and make a comparative calculation.

Hereinafter, as the case may be, the following method will be referred to as the “LED/photodiode method”: a method using the measuring apparatus 10 of the embodiment, wherein the light-emitting elements 25 (LEDs) of the predetermined wavelengths are used as light sources, to measure the voltage generated when light going through a measurement sample is received by the light-receiving element 32 (photodiode), and then obtaining the concentration in the measurement sample on the basis of the measured value.

### Example 2

In Example 2, appropriate amounts of distilled water were each added to the test solution in Example 1. In this way, peracetic acid solutions having four concentrations different from each other (samples 1 to 4) were prepared. By iodometric titration, each of the peracetic acid concentrations was quantitatively determined two times.

From each of the samples 1 to 4, a fraction having a volume of 0.2 mL was taken out, and thereto was added 240 mg/L of a potassium iodide solution to set the total volume to 20 mL (measurement samples). A light-emitting diode (wavelength: 470 nm) and a photodiode were used to measure the voltage generated therefrom two times. About each of the samples, the peracetic acid concentration in the sample was calculated from the measured voltage and the regression formula “y=−0.0119x+0.9326” shown in FIG. 2. As shown in Table 1, the measured results according to the iodometric titration and those according to the LED (470 nm)/photodiode method were very close to each other.

TABLE 1
Comparison of measured results of the peracetic acid concentration
according to iodometric titration with those according to
LED/photodiode method

| | | | LED (470 nm)/ | |
|---|---|---|---|---|
| | | | photodiode method | |
| | | Iodometric | Concen- | |
| | | titration | tration in | |
| | | Concentration | measurement | |
| | | in | sample | Concentration |
| | | sample (%) | (ppm) | in sample (%) |
| Sample 1 | First time | 0.295 | 29.7 | 0.297 |
| | Second time | 0.290 | 29.6 | 0.296 |
| Sample 2 | First time | 0.244 | 24.5 | 0.245 |
| | Second time | 0.242 | 24.5 | 0.245 |
| Sample 3 | First time | 0.199 | 20.3 | 0.203 |
| | Second time | 0.200 | 20.1 | 0.201 |
| Sample 4 | First time | 0.188 | 18.8 | 0.188 |
| | Second time | 0.186 | 18.9 | 0.189 |

### Example 3

In Example 3, a 6%-peracetic acid antiseptic solution (trade name: ACECIDE manufactured by Saraya Co., Ltd. diluted 20 times with distilled water, and the resultant was used as a test solution. To 0.2 mL of the test solution were added 240 mg/L of a potassium iodide solution and each of 0 mL, 1 mL and volumes therebetween of 1% hydrogen peroxide water to set the total volume to 20 mL (measurement samples). The LED (470 nm)/photodiode method was used to measure each of the samples three times. On the basis of the approximately straight line in FIG. 2, results obtained by calculating the peracetic acid concentration in the test solution are shown in Table 2.

TABLE 2
Effects on measured values produced by the hydrogen peroxide amount

| | | Added amount of hydrogen peroxide | | | |
|---|---|---|---|---|---|
| | | 0 mL | 0.1 mL | 0.5 mL | 1.0 mL |
| Hydrogen peroxide amount in | | 0.026 mmol | 0.056 mmol | 0.173 mmol | 0.320 mmol |
| measurement sample | | | | | |
| Measured values | First time | 0.516 V | 0.519 V | 0.512 V | 0.510 V |
| | Second time | 0.516 V | 0.519 V | 0.514 V | 0.509 V |
| | Third time | 0.516 V | 0.520 V | 0.515 V | 0.511 V |
| Peracetic acid concentration | First time | 0.350% | 0.348% | 0.353% | 0.355% |
| in test solution calculated | Second time | 0.350% | 0.348% | 0.352% | 0.356% |
| from FIG. 2 | Third time | 0.350% | 0.347% | 0.351% | 0.354% |

This test solution concentration was quantitatively determined by iodometric titration. As a result, the concentration of peracetic acid was 0.356% and that of hydrogen peroxide was 0.446%. In the measurement sample wherein this was used in an amount of 0.2 mL, the amount of peracetic acid was 0.0094 mmol, and that of hydrogen peroxide was 0.026 mmol. As shown in Table 2, even when hydrogen peroxide was added to this to increase the total amount thereof to 0.320 mmol, the measured value and the calculated concentration of peracetic acid in the test solution were hardly affected.

### Example 4

In Example 4, 240 mg/L of a potassium iodide solution was added to each of 0.2 mL, 1.5 mL and volumes therebetween of a 0.03% perpropionic acid solution to prepare a measurement sample having a total volume of 20 mL. The LED (470 nm)/photodiode method was then used to measure the voltage therein.

As illustrated in FIG. 3, about the percarboxylic acid other than peracetic acid, a linear relationship was obtained between the concentrations of the percarboxylic acid and the measured values (voltage values: V).

### Example 5

In Example 5, 240 mg/L or 480 mg/L of a potassium iodide solution was added to each of 0.05 mL, 1.0 mL and volumes therebetween of peracetic acid (the concentration of peracetic acid was 0.355% according to iodometric titration) to prepare a measurement sample having a total volume of 20 mL. The LED (470 nm)/photodiode method was then used to measure the voltage therein. The measured results are shown in FIG. 4.

In the cases wherein the peracetic acid amount was from 0.05 to 0.3 mL (corresponding to peracetic acid concentrations of 8.9 to 53 ppm in the measurement samples), a linear relationship was recognized between the peracetic acid concentrations (ppm) in the measurement samples and the measured values (voltage values: V). However, in the cases where the amount was 0.4 mL (corresponding to a peracetic acid concentration of 71 ppm in one of the measurement samples) or more, the measured values (V) deviated from a straight line about each of the potassium iodide concentrations.

### Example 6

In Example 6, an effect produced by the pH of measurement samples was examined. To 0.2 mL of about 0.35% peracetic acid was added each of potassium iodide solutions which were each 240 mg/L in volume and had pHs of 3, 6 and values therebetween, respectively, the pHs being adjusted with citric acid and sodium citrate. In this way, each measurement sample having a total volume of 20 mL was prepared. The LED (470 nm)/photodiode method was then used to measure the voltage therein. A change in the voltage value over 10 minutes from the end of the preparation of each of the measurement samples is shown in FIG. 5. To 0.05 mL of a 2% hydrogen peroxide solution was added each of potassium iodide solutions each having a concentration of 240 mg/L and having adjusted pHs of 1, 3 and a value therebetween, respectively, in order to give a concentration equal or similar to that (0.4%) of hydrogen peroxide contained in 0.2 mL of 0.35% peracetic acid. In this way, each measurement sample having a total volume of 20 mL was prepared. The LED (470 nm)/photodiode method was then used to measure the voltage therein over 10 minutes, and a change in the voltage value is shown in FIG. 6.

According to FIG. 5, the measured values were stable in the pH range of 3 to 5; however, when the pH value was turned to 6, the measured values were unstable. Moreover, according to FIG. 6, when the pH value was lowered to 1, the measured values were unstable since hydrogen peroxide and potassium iodide reacted slowly with each other to generate iodine.

### Example 7

In Example 7, an effect of the amount of potassium iodide was examined. To 0.2 mL (0.0094 mmol) of a 0.331% peracetic acid solution was added each of potassium iodide solutions the mole numbers of which were 2, 3, 15, 30, 60 and 90 times that of the acid, respectively. In this way, each measurement sample having a total volume of 20 mL was prepared. For example, about the potassium iodide solution wherein the mole number of the iodide was 90 times (90 KI), 0.84 mmol of potassium iodide was contained in the solution having a volume of 20 mL. The absorbance was measured at 470 nm, and a change in the absorbance after the preparation of each of the measurement samples is shown in FIG. 7.

When the concentration of potassium iodide is largely excessive, the measured value changes with time. Thus, it is necessary to make the measurement immediately after each of the test solutions is prepared. However, when about the concentration of potassium iodide, the mole number thereof was from 3 to 15 times that of peracetic acid, a change was hardly caused with time. As a result, stable measured values were able to be obtained.

### INDUSTRIAL APPLICABILITY

The present invention can be effectively used in the case of measuring only the concentration of a percarboxylic acid easily and quickly in an equilibrium mixture containing the percarboxylic acid and hydrogen peroxide.

1. A method for measuring only a concentration of a peroxycarboxylic acid in an equilibrium mixture containing peroxycarboxylic acid and hydrogen peroxide, comprising the following steps a) and b);
a) adding potassium iodide to the equilibrium mixture to cause the generation of iodine and providing the resulting mixture as a measurement sample; and
b) measuring the amount of light that goes through the measurement sample to determine only the concentration of a peroxycarboxylic acid.

2. The method for measuring according to claim 1, wherein the concentration of a peroxycarboxylic acid in the measurement sample is within a range of 0.01 to 50 ppm.

3. The method for measuring according to claim 1, wherein the pH value of the measurement sample is within a range of 1<pH<6.

4. The method for measuring according to claim 1, wherein the amount of potassium iodide in the measurement sample is within a range of 2 to 60 times of the number of moles of the peroxycarboxylic acid.

5. The method for measuring according to claim 1, wherein the wavelength of the light used in the measurement is within a range of 440 to 600 nm.

6. The method for measuring according to claim 1, wherein the peroxycarboxylic acid is peracetic acid.

7-8. (canceled)

9. The method for measuring according to claim 2, wherein the pH value of the measurement sample is within a range of 1<pH<6.

10. The method for measuring according to claim 2, wherein the amount of potassium iodide in the measurement sample is within a range of 2 to 60 times of the number of moles of the peroxycarboxylic acid.

11. The method for measuring according to claim 3, wherein the amount of potassium iodide in the measurement sample is within a range of 2 to 60 times of the number of moles of the peroxycarboxylic acid.

12. The method for measuring according to claim 2, wherein the wavelength of the light used in the measurement is within a range of 440 to 600 nm.

13. The method for measuring according to claim 3, wherein the wavelength of the light used in the measurement is within a range of 440 to 600 nm.

14. The method for measuring according to claim 4, wherein the wavelength of the light used in the measurement is within a range of 440 to 600 nm.

15. The method for measuring according to claim 2, wherein the peroxycarboxylic acid is peracetic acid.

16. The method for measuring according to claim 3, wherein the peroxycarboxylic acid is peracetic acid.

17. The method for measuring according to claim 4, wherein the peroxycarboxylic acid is peracetic acid.

18. The method for measuring according to claim 5, wherein the peroxycarboxylic acid is peracetic acid.
